(19) United States
(12) Patent Application Publication
(10) Pub. No.: US 2026/0128136 A1
(43) Pub. Date: May 7, 2026

(54) TECHNIQUES FOR SUBSTITUTING INFORMATION INTO ORDERS PRIOR TO PERSISTENCE IN STORAGE OF A DATA RECORD SYSTEM

(71) Applicant: Flatiron Health, Inc., New York, NY (US)

(72) Inventors: Kyle Ritter, Carthage, TX (US); Shawn Huda, New York, NY (US); Andrea Herrera, New York, NY (US); Alexa Gleason Hibbert, New York, NY (US); Ina Ochoche, Gaithersburg, MD (US); Marlon Fischer, East Meadow, NY (US); Jesse Watts-Russell, Los Angeles, CA (US); David Brandon, Sherwood, AR (US); Yael Stochel, New York, NY (US); Chris Rovillos, New York, NY (US); Helena Hay, Brooklyn, NY (US); Emma Vail, New York, NY (US); Maya Flannery, Brooklyn, NY (US); Minh Nguyen, Brooklyn, NY (US); Enrique Alonso, New York, NY (US); Jake Lovitz, Forest Hills, NY (US); Ivan Quan, Jersey City, NJ (US)

(73) Assignee: Flatiron Health, Inc., New York, NY (US)

(21) Appl. No.: 18/940,512

(22) Filed: Nov. 7, 2024

(51) Int. Cl. G16H 10/60; G16H 70/40

(52) U.S. Cl. CPC G16H 10/60; G16H 70/40

(57) ABSTRACT

Described herein are techniques for automatically substituting information into orders generated by a data record system prior to persisting the orders in storage of the data record system. The techniques store substitution groups of data entities that store data associated with respective substances (e.g., medications). When order templates (e.g., medication regiments) are generated by the data record system, the techniques automatically modify the order templates (e.g., by substituting data entities in the orders) based on data entity substitution configurations of the substitution groups. Information from the modified order templates is input into orders before the orders are persisted in storage of the data record system.

### BACKGROUND

Orders may be generated and persisted in storage of a data record system. One example of a data record system is an electronic health record (EHR) system. An EHR may generate medication orders for patients and persist them in storage of the EHR. For example, a physician may use a graphical user interface (GUI) provided by the EHR to submit information specifying parameters of a medication order (e.g., one or more medications to be included in the medication order, instructions for administering the medication(s) to a patient, a time interval at which to administer the medication(s) to the patient, and/or other information). The EHR system may generate an order using the information received through the GUI and persist the order in storage of the EHR system (e.g., by storing the medication order in a storage location associated with a particular patient's health record). The medication(s) indicated by the order may then be administered to a patient based on the information specified in the order.

Another example of a data record system is a transaction database used by an e-commerce software application. The software application may generate orders (e.g., resulting from transactions in the software application) and persist the orders in storage of the data record system. The software application may generate an order using information obtained through a GUI provided by the software application (e.g., payment information, customer identification information, specification of item(s) to include in the order) and persist the order in the transaction database (e.g., by storing the order in a queue of orders to be fulfilled). The order may then be fulfilled (e.g., by packaging and shipping item(s) specified by the order).

### SUMMARY

Described herein are techniques for automatically substituting information into orders generated by a data record system prior to persisting the orders in storage of the data record system. The techniques store substitution groups of data entities that store data associated with respective substances (e.g., medications). When order templates (e.g., medication regiments) are generated by the data record system, the techniques automatically modify the order templates (e.g., by substituting data entities in the orders) based on data entity substitution configurations of the substitution groups. Information from the modified order templates is input into orders before the orders are persisted in storage of the data record system.

Some embodiments provide a data processing system for automatically substituting information into orders prior to persisting the orders in storage of a data record system, the data record system storing a plurality of data entities storing data associated with a plurality of substances. The data processing system comprises: a computer hardware processor; and memory storing: data indicating a plurality of substitution groups each assigned a subset of the plurality of data entities and including a data entity substitution configuration, the substitution groups including a first substitution group assigned a first subset of data entities sharing a common active ingredient of respective substances; and instructions that, when executed by the computer hardware processor, cause the computer hardware processor to: obtain a first order template generated by the data record system, the first order template indicating a first data entity of the first subset of data entities assigned to the first substitution group; access a first data entity substitution configuration of the first substitution group, the first data entity substitution configuration indicating an instruction to substitute a second data entity of the first subset of data entities in place of the first data entity; modify the first order template based on the first data entity substitution configuration at least in part by replacing the first data entity indicated by the first order with the second data entity to obtain a modified first order template; and persist, in the storage of the data record system, a first order storing information from the second data entity indicated by the modified first order template.

Some embodiments provide a method for automatically substituting information into orders prior to persisting the orders in storage of a data record system, the data record system storing a plurality of data entities storing data associated with a plurality of substances. The method comprises using a computer hardware processor to perform: obtaining a first order template generated by the data record system, the first order template indicating a first data entity of a first subset of data entities assigned to a first substitution group of a plurality of substitution groups, the first subset of data entities sharing a common active ingredient of respective substances; accessing a first data entity substitution configuration of the first substitution group, the first data entity substitution configuration indicating an instruction to substitute a second data entity of the first subset of data entities in place of the first data entity; modifying the first order template based on the first data entity substitution configuration at least in part by replacing the first data entity indicated by the first order with the second data entity to obtain a modified first order template; and persisting, in the storage of the data record system, a first order storing information from the second data entity indicated by the modified first order template.

Some embodiments provide a computer-readable medium storing instructions that, when executed by a computer hardware processor, cause the computer hardware processor to perform a method for automatically substituting information into orders prior to persisting the orders in storage of a data record system, the data record system storing a plurality of data entities storing data associated with a plurality of substances. The method comprises: obtaining a first order template generated by the data record system, the first order template indicating a first data entity of a first subset of data entities assigned to a first substitution group of a plurality of substitution groups, the first subset of data entities sharing a common active ingredient of respective substances; accessing a first data entity substitution configuration of the first substitution group, the first data entity substitution configuration indicating an instruction to substitute a second data entity of the first subset of data entities in place of the first data entity;

modifying the first order template based on the first data entity substitution configuration at least in part by replacing the first data entity indicated by the first order with the second data entity to obtain an updated first order template; and persisting, in the storage of the data record system, a first order storing information from the second data entity indicated by the modified first order template.

The foregoing summary is non-limiting.

### BRIEF DESCRIPTION OF DRAWINGS

Various aspects and embodiments will be described with reference to the following figures. It should be appreciated that the figures are not necessarily drawn to scale. Items appearing in multiple figures are indicated by the same or a similar reference number in all the figures in which they appear.

FIG. 1 shows an example data record system generating and persisting orders.

FIG. 2 shows an example data record system including a data processing system, according to some embodiments of the technology described herein.

FIG. 3A shows an example of mapping a set of data entities to a reference data entity based on a shared active ingredient of substances associated with the set of data entities and the reference data entity, according to some embodiments of the technology described herein.

FIG. 3B shows another example of generating a collection of data entities associated with biologic substances, according to some embodiments of the technology described herein.

FIG. 4 shows an example of defining a substitution group, according to some embodiments of the technology described herein.

FIG. 5 shows an example of generating a data entity substitution group configuration for a substitution group, according to some embodiments of the technology described herein.

FIG. 6A illustrates a data entity indicated by an order template being identified as belonging to a substitution group, according to some embodiments of the technology described herein.

FIG. 6B illustrates substituting of another data entity in place of the data entity identified as belonging to the substitution group in FIG. 6A based on a data entity substitution group of the substitution group, according to some embodiments of the technology described herein.

FIG. 7 illustrates a process for automatically substituting data entities in order templates generated by a data record system prior to persisting the order templates as orders in storage of the data record system, according to some embodiments of the technology described herein.

FIG. 8 shows a GUI for generating a new substitution group, according to some embodiments of the technology described herein.

FIG. 9A shows a GUI for specifying a data entity substitution preference of a data entity substitution configuration of a substitution group, according to some embodiments of the technology described herein.

FIG. 9B shows a GUI for specifying delivery overrides of the data entity substitution configuration of FIG. 9A, according to some embodiments of the technology described herein.

FIG. 10 shows a GUI for creating an order template, according to some embodiments of the technology described herein.

FIG. 11 shows a GUI displaying information about a persisted order, according to some embodiments of the technology described herein.

FIG. 12 shows a block diagram of an exemplary computing device that may be specially configured to implement some embodiments of the technology described herein.

### DETAILED DESCRIPTION

The inventors have developed techniques for automatically substituting information into orders generated by a data record system prior to persisting the orders in storage of the data record system. The substitutions may automatically modify undesired information in the orders and/or apply custom preferences to the orders during the generation of the data records (as opposed to modifying orders after they have persisted in storage). For example, some embodiments may automatically substitute medication(s) into a medication order generated in an EHR system prior to the persistence of the medication order in storage of the EHR system for administration of medication(s) to patient(s).

An EHR system is used by medical institutions (e.g., hospitals, clinics, medical practices, labs, and/or other medical institutions) to store data records for patients (e.g., digital versions of a patient's health history). In addition to storing data records, the EHR system may provide various types of functionalities that facilitate the operations of a medical institution such as generating orders for patients indicating one or more substances (e.g., medication(s)) to be administered to patients and instructions for doing so. For example, the EHR system may provide a GUI through which a care provider (e.g., a physician) can specify information for an order (e.g., one or more medications to be administered to a patient and instructions for administering the medication(s)). An order generated by the EHR system may be persisted in storage of the EHR (e.g., in a digital chart of the patient stored by the EHR). A substance (e.g., a medication) indicated by the order may then be administered to the patient according to instructions specified by the order.

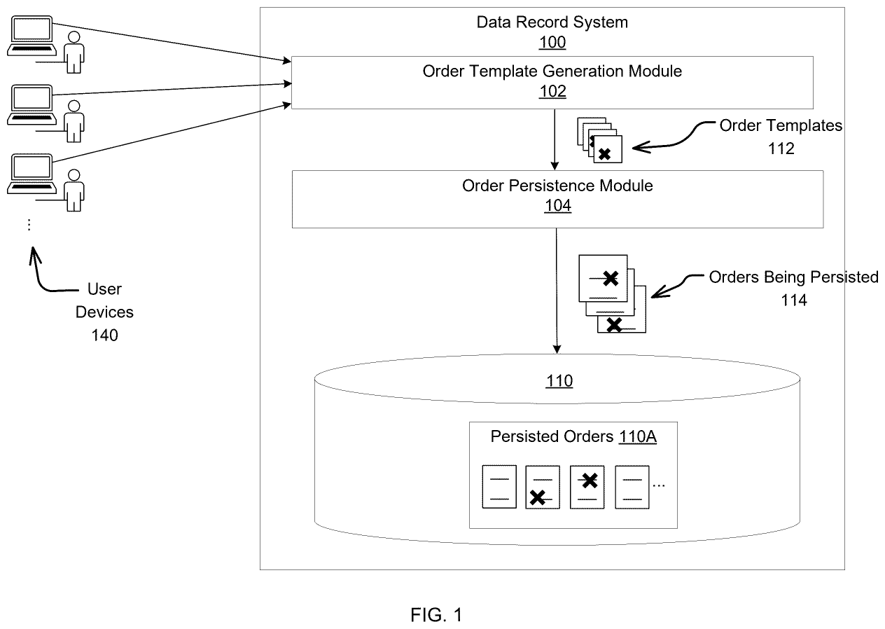

FIG. 1 shows an example of a data record system 100 (e.g., an EHR system) generating and persisting orders. As shown in FIG. 1, the data record system 100 includes an order template generation module 102 configured to generate order templates 112. An order template may indicate information that is used to generate one or more orders. For example, in an EHR system, an order template may indicate a regimen of one or more medications for a subject (e.g., a patient). The order template generation module 102 may be configured to generate order templates 112 based on input from user devices 140. The order template generation module 102 may be configured to obtain information from the user devices 140 for orders to be generated and store the information in the order templates 112. For example, for an EHR system, the order template generation module 102 may obtain information about medication orders to be generated through graphical user interfaces (GUIs) provided on the user devices 140. Care providers may enter information through the GUIs. The order template generation module 102 may store the information obtained through the GUIs in data fields of the order templates 112.

The data record system 100 further includes an order persistence module 104 configured to persist orders 114 in data storage 110 (e.g., a database) of the data record system 100. The order persistence module 104 may be configured to persist the orders 114 by: (1) writing information from the order templates 112 into respective data records, and (2) persisting the data records in the data storage 110 as orders 114 in the data storage. For example, for an EHR system, the order persistence module 104 may store information from order templates in medication orders and persist the medication orders in the appropriate patient charts.

The inventors have recognized a problem in the operation of data record systems such as EHR systems. Orders generated and persisted in the data storage 110 may often include undesired information. The undesired information may have been included in the orders based on user input obtained by the order template generation module 102 and stored in order templates 112 from which orders are generated and subsequently persisted in data storage 110. In the context of an EHR system, one challenge that leads to orders (e.g. medication orders) with undesired information is that there are multiple medications that share a common active ingredient, and which can be prescribed to a patient in a medication order. When creating a new order in an EHR system, a user (e.g., a physician) may specify one of multiple medications that share a common active ingredient. However, there are various reasons that the medication specified by the user may not be desired. For example, the cost of the specified medication may not be affordable to a patient, the specified medication may not be available (e.g., out of stock), and/or the health insurance of the patient may not cover the specified medication. As another example, a medical institution may have a preference or requirement to use a particular medication over another medication (e.g., due to availability, costs, and/or other factors).

As illustrated in FIG. 1, the order templates 112 generated by the order template generation module 102 include undesired information (indicated by the “x” marks in the order templates 112) (e.g., due to input by users of the devices 140). The order template generation module 102 transmits the order templates 112 with undesired information (e.g., undesired medications) to the order persistence module 104 which in turn persists orders 114 into the storage 110. The above-described problem leads to various issues in an EHR system because medication orders persisted in patient charts may lead to undesired medications being administered to patients, delays in medication being administered to patients, and even failure to administer medication to patients. Moreover, the orders including undesired information require additional processing by the data record system 100 to update the orders.

Conventional EHR systems address the above-described problem by updating a persisted order when issue with information in the order (e.g., an undesired medication is specified in the order) is recognized. However, this requires significant amounts of computation to update orders. When orders (e.g., medication orders that indicate undesired medications) need to be updated, the EHR system is required to perform significant additional data processing. The EHR system needs to access a persisted order from storage, obtain information for updating the order from one or more user devices (e.g., through GUI(s)), delete prior data stored in the order, write additional data into the order, and persist the updated order in storage. Moreover, an EHR system is completely reliant on a user recognizing an issue with information in an order to resolve the issue (e.g., by updating the order to modify information therein). Thus, the EHR system may continue to store several medical orders that specify undesired medications without any capability to resolve the issues.

To address the above-described problem in data record systems (e.g., EHR systems), the inventors have developed systems and techniques that automatically configures orders at the point of order generation. The techniques mitigate the risk of undesired information being introduced into an order during generation of the order and prior to persistence of the order in storage. During generation of an order, the system: (1) recognizes that a particular substance (e.g., a medication) indicated by an order template needs to be substituted, (2) automatically modifies the order template by substituting the substance indicated by the order template, and (3) persists an order in data storage using the modified order template. For example, in the context of an EHR system, the system recognizes that a medication specified in an order template needs to be substituted with another medication (e.g., due to supply of the medication, preference of a medical entity, cost of the medication, insurance coverage of the medication, and/or another reason) and substitutes the medication into the order prior to the medication order being persisted in data storage of the EHR system (e.g., in a digital patient chart).

The systems and techniques described herein improve technology of data record systems (e.g., EHR systems) by reducing the database processing (e.g., reading orders from storage, deleting data from the orders, and/or writing data to the orders) that needs to be performed to update previously persisted orders (e.g., when a care provider specifies a medication for a medication order). Moreover, by performing substitutions in real-time at the point orders are generated, the systems and techniques reduce the amount of undesired information in orders persisted in data storage. Accordingly, embodiments described herein improve technology of data record systems such as EHR systems.

Some embodiments provide a data processing system for automatically substituting data entities into orders generated by a data record system prior to persisting the orders in storage of the data processing system. The data record system may store data entities that each store information associated with a respective substance (e.g., a medication) that may be indicated in an order generated by the data record system. The information may include identification of the substance, an active ingredient in the substance, a delivery route for the substance to be administered to a patient, and/or other information about the substance. The system may be configured to store data indicating substitution groups that are each assigned a subset of the data entities and include a data entity substitution configuration. The substitution groups may include a substitution group assigned a subset of the data entities that share a common active ingredient of associated substances. The system may be configured to obtain an order template generated by a data record system (e.g., an EHR system) that indicates a first data entity (e.g., associated with a first medication). The first data entity may be assigned to the substitution group. The system may be configured to access a data entity substitution configuration associated with the substitution group that indicates an instruction to substitute a second data entity (e.g., associated with a second medication) from the substitution group in place of the first data entity. The system may be configured to modify the order template based on the data entity substitution configuration by replacing the first data entity indicated by the order template with the second data entity. The system may be configured to persist an order storing information from the modified order template (e.g., from the second data entity indicated by the modified order template) in memory of the data record system.

In some embodiments, the system may be configured to generate the first substitution group by performing: (1) access a reference data entity storing information about a reference substance (e.g., a reference medication), the reference data entity indicating a particular active ingredient of the reference substance, (2) identify, from among the plurality of data entities, data entities based on the reference data entity, the identifying comprising identifying data entities that indicate the particular active ingredient of the reference substance (e.g., by identifying data entities that are mapped to the reference data entity), and (3) assign at least some of the identified data entities to the substitution group (e.g., by receiving user input selecting at the at least some data entities through a GUI). In some embodiments, the reference data entity further indicates a particular delivery route of the reference substance and identifying, from among the plurality of data entities, the data entities based on the reference data entity further comprises identifying data entities that indicate the particular delivery route of the reference substance in addition to the particular active ingredient of the reference substance.

In some embodiments, the data entity substitution configuration further comprises a delivery override, the delivery override indicating that a delivery method corresponding to the first data entity is to be overridden by a second delivery method when the second data entity is substituted into an order template. In some embodiments, the system may be configured to modify the order template based on the data entity configuration associated with the substitution group at least in part by changing the first delivery method to the second delivery method. In some embodiments, the data entity substitution configuration further includes a data entity override that overrides the instruction to substitute the second data entity in place of the first data entity, wherein the data entity override specifies a particular data entity to use when a target subject (e.g., a patient) in an order template has a particular attribute (e.g., has a particular insurance provider). In some embodiments, the system may be configured to: (1) obtain a second order template generated by the data record system for a first target subject, the second order template indicating the first data entity of the first subset of data entities assigned to the substitution group, (2) access the data entity substitution configuration associated with the substitution group, and (3) modify the second order template based on the first data entity substitution configuration. The system may be configured to modify the second order template at least in part by: (1) determining that the first target subject has the particular attribute specified by the data entity override of the data entity substitution configuration, and (2) when it is determined that the first target subject has the particular attribute specified by the data entity override, replacing the first data entity indicated by the second order template with the particular data entity to obtain a modified second order template. The system may be configured to persist, in the storage of the data record system, a second order storing information from the particular data entity indicated by the modified second order template.

In some embodiments, the substitution groups include a substitution group assigned a subset of the plurality of data entities that store data associated with a set of biosimilar substances (e.g., a set of biosimilar medications), the substitution group may have a data entity substitution configuration. The system may be configured to: (1) obtain a second order template generated by the data record system, the second order template indicating a third data entity of the second subset of data entities assigned to the biosimilar substitution group, (2) access a data entity substitution configuration associated with the biosimilar substitution group, the data entity substitution configuration indicating an instruction to substitute a fourth data entity of the subset of data entities assigned to the biosimilar substitution group in place of the third data entity, (3) modify the second order template based on the data entity substitution configuration at least in part by replacing the third data entity indicated by the second order template with the fourth data entity to obtain a modified second order template, and (4) persist, in the data storage of the data record system, a second order storing information from the fourth data entity indicated by the modified second order template.

In some embodiments, the data entity substitution configuration of the biosimilar substitution group further includes a data entity override that overrides the instruction to substitute the fourth data entity in place of the third data entity, wherein the data entity override specifies a particular data entity to use when a target subject in an order template has a particular attribute.

In some embodiments, the system may be configured to update previously persisted orders stored in data storage of a data record system. The system may be configured to: (1) recognize that a particular substance (e.g., a medication) indicated in an order is to be substituted, (2) automatically modifies the order by substituting the substance indicated by the order, and (3) persists the updated order in data storage. The system may be configured to access a substitution group that the particular substance belongs to (e.g., that a data entity associated with the particular substance is assigned to) and modify the order using a data entity substitution configuration of the substitution group. For example, the system may determine, based on the data entity substitution configuration, that the particular substance is to be substituted with another substance. The system may access information about the other substance from a data entity associated with the other substance and update the order using the information (e.g., by replacing information in the order with the information from the data entity associated with the other substance).

Following below are more detailed descriptions of various concepts related to, and embodiments of, substituting correct information into newly generated orders prior to the orders being persisted in storage of a data record system. It should be appreciated that various aspects described herein may be implemented in any of numerous ways. Examples of specific implementations are provided herein for illustrative purposes only. In addition, the various aspects described in the embodiments below may be used alone or in any combination and are not limited to the combinations explicitly described herein. Some example embodiments are described herein in the context of a data record system comprising an EHR system for illustrative purposes. However, the embodiments are not restricted to EHR systems. Some embodiments may be implemented in other types of data record systems.

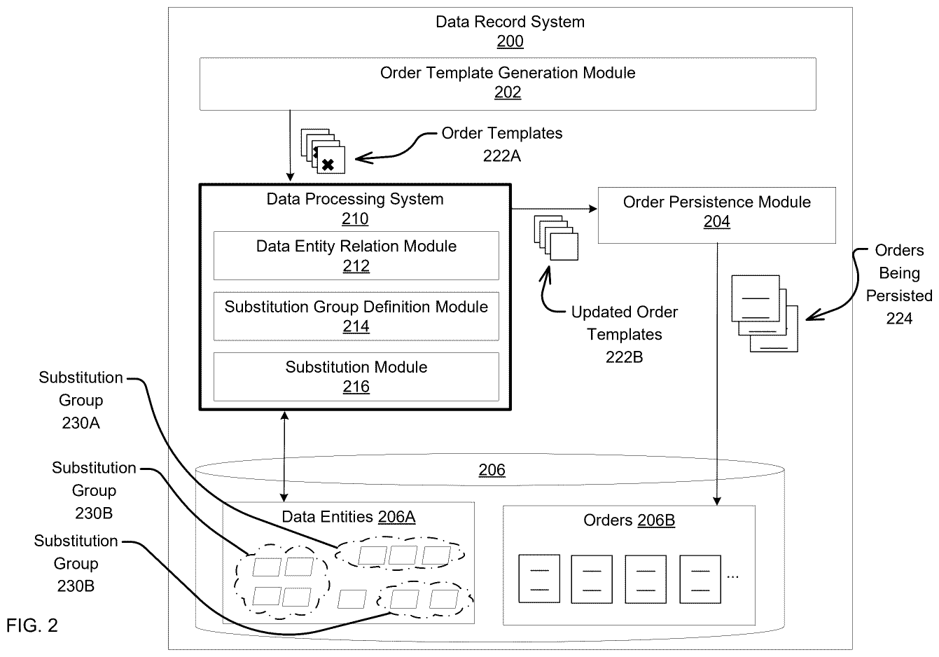

FIG. 2 shows an example data record system 200 including a data processing system 210, according to some embodiments of the technology described herein. As illustrated in FIG. 2 the data processing system 210 automatically applies updates during the generation of orders prior to the persistence of the orders in storage 206 of the data record system 200. By doing so the data processing system 210 improves the accuracy of orders persisted in the storage 206 of the data record system 200. For example, in implementations in which the data record system 200 is an EHR system, the data processing system 210 may apply updates during the generation of medication orders that automatically substitute medications into orders in place of other medications (e.g., based on a configuration of preferences of a medical institution). This reduces the amount of undesired information in medication orders that need to be resolved by the EHR system (e.g., by modifying medication orders in the data storage).

As shown in FIG. 2, the data record system 200 includes an order template generation module 202, an order persistence module 204, data storage 206, and the data processing system 210. The order temperate generation module 202 generates order templates 222A from which orders are generated and persisted into data storage 206 by the order persistence module 204. The data processing system 210 modifies the order templates 222A to generate updated order templates 222B that are then used by the order persistence module 204 to persist orders 224 into the data storage 206. As shown in FIG. 2, the order templates 222A include data entities (indicated by the “x” marks) that are substituted with other data entities in the order templates 222B as a result of the modifications applied by the data processing system 210.

As shown in FIG. 2, the data storage 206 of the data record system 200 stores data entities 206A. The data entities 206A may store information associated with items that can be specified in an order template (e.g., generated by the order template generation module 202). Each data entity may store information associated with a particular item. In some embodiments, the items may be substances (e.g., medications) that can be administered to humans (e.g., patients). Each of the data entities 206A may store information associated with a substance. In some embodiments, each of the data entities 206A may store an active ingredient of the substance and a delivery route through which the substance is administered. The data entities 206A may store additional information associated with the substance such as identification information (e.g., a name, ID, class, and/or other identification information), availability of the substance, dosing information (e.g., amount of fluid, dose limits, lifetime of dose, length of dose, and/or other dosing information), administration information (e.g., instructions, link to prescription, and/or other administration information), and/or other information associated with the substance.

The data storage 206 of the data records system 200 stores orders 206B (e.g., that have been persisted in memory by the order persistence module 204). In some embodiments, an order may indicate a substance to be administered to an individual (e.g., a patient). In some embodiments, an order may indicate information for administering the substance to an individual. For example, an order may indicate a medication to be administered to a patient and instructions for administrating the medication to the patient (e.g., route, fluid type, amount, duration, start date, end date, and/or other information). An order may indicate other information such as a date, a diagnosis of the patient (e.g., a type of cancer the patient is diagnosed with and/or a stage of cancer), a location, an identification of a user (e.g., care provider) that created the order, information about the patient (e.g., weight, height, and/or other information) and/or other information.

The data storage 206 of the data record system 200 may comprise any suitable storage hardware. For example, the data storage 206 may include one or more hard drives (e.g., solid state drives (SSDs), hard disk drives (HDDs), and/or other types of hard drives). In some embodiments, the data storage 206 may comprise a database configured to store the data entities 206A and the orders 206B. For example, for embodiments in which the data record system 200 is an EHR system, the data storage 206 may comprise a database storing patient medical records. For example, the database may store a medical record of each patient in a patient chart. The orders 206B persisted in the data storage 206 may be stored in association with the patient medical records. For example, the orders 206B may be medication orders prescribing a medication regimen for the patients. The orders 206B may thus be included in respective patient medical records. In some embodiments, persisting an order (e.g., a medication order) in a patient's medical record may trigger administration of medication(s) to the patient in accordance with the order.

As shown in FIG. 2, the order template generation module 202 may be configured to generate order templates 222A specifying information from which orders are generated. In some embodiments, the order template generation module 202 may be configured to generate an order template by: (1) obtaining information from a user (e.g., through a GUI provided by the data record system 200 to the user's device as described herein with reference to FIG. 1), and (2) generating the order template using the information from the user. For example, the order template generation module 202 may obtain input from a user indicating a substance (e.g., a medication to include in an order (e.g., a medication order), and store the information in an order template. Information obtained from the user may include an indication of a substance to include in the order, administration instructions (e.g., route, fluid type, amount, duration, start date, and/or end date), and/or other information. In some embodiments, information obtained from the user may include other information such as a date of creating the order template, a facility location, a patient diagnosis, billing code(s), and/or an identification of a care provider (e.g., a physician) submitting the information.

In some embodiments, an order template generated by the order template generation module 202 (e.g., using information obtained from a user) may indicate one or more data entities associated with respective substance(s). For example, the order template may indicate one or more data entities associated with medication(s) that are to be administered to a patient (e.g., specified by user input received through a GUI provided by the data record system 200). In some embodiments, the order template may further indicate administration instructions for administering each of the substance(s). For example, the order template may indicate a fluid amount, duration, and/or other administration instructions for each substance.

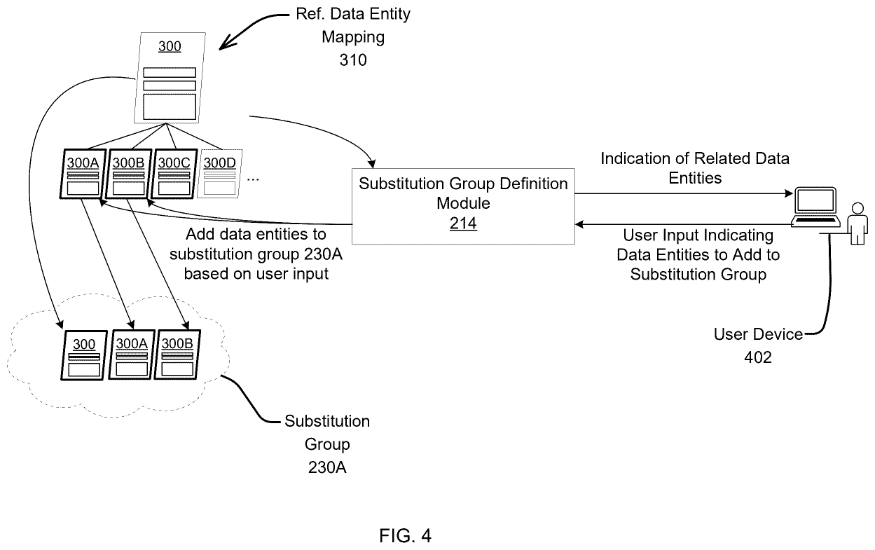

As shown in FIG. 2, the order templates 222A generated by the order template generation module 222A are processed by the data processing system 210 which applies updates to the order templates 222A to obtain updated order templates. In some embodiments, the data processing system 210 may be configured to update the order templates 222A generated by the order template generation module 202 by substituting data entities into the orders. The data processing system 210 may be configured to substitute data entities into orders by using substitution groups. A substitution group is a group of data entities associated with respective substances that can be substituted in place of each other in an order template. The data processing system 210 may be configured to define substitution groups. Example techniques for defining a substitution group are described herein with reference to FIG. 4. For example, the data processing system 210 may define a substitution group by: (1) receiving user input indicating a selection of data entities to include in the substitution group, and (2) defining the substitution group that includes the indicated data entities. In the example of FIG. 2, substitution groups 230A, 230B, 230C have been defined by the data processing system 210.

In some embodiments, a substitution group may be a group of data entities associated with substances that each share a common active ingredient. Each of the data entities may indicate the common active ingredient shared by the substances. In some embodiments, the group of data entities may further indicate the same delivery route in addition to the common active ingredient. In some embodiments, a substitution group may be a group of data entities associated with a set of biosimilars (i.e., a group of biologic substances that have no clinically meaning differences in safety, purity, or potency).

In some embodiments, a substitution group may be defined by a reference data entity. Other data entities may be assigned to the substitution group based on the reference data entity. For example, the other data entities may indicate active ingredients that are indicated by the reference data entity. As another example, the other data entities may further indicate the same delivery route as the reference data entity. In some embodiments, the substitution group may comprise of data entities that are associated with substances that are biosimilar to a substance associated with a reference data entity. In some embodiments, the substitution groups may be mutually exclusive. Thus, a data entity in one substitution group may not belong to another substitution group. In some embodiments, the substitution groups may not be mutually exclusive. A data entity in one substitution group may belong to another substitution group.

In some embodiments, each of the substitution groups 230A, 230B, 230C may include a data entity substitution configuration. The data entity substitution configuration may indicate instructions to substitute a first data entity of the substance group associated with a first substance with a second data entity of the substance group associated with a second substance. For example, the data entity substitution configuration may include a preference of data entities (e.g., a ranking of data entity preference). As another example, the data entity substitution configuration may include overrides based on subject attributes (e.g., patient attributes). To illustrate, the data entity substitution configuration may specify an instruction to override a preferred data entity from the substitution group with another data entity when the subject has a particular insurance provider (e.g., because the insurance provider may only reimburse for the substance associated with the other data entity). The data processing system 210 may be configured to perform substitutions based on data entity substitution configurations of substitution groups.

The data processing system 210 may be configured to apply updates to the order templates 222A using the substitution groups 230A, 230B, 230C. When the data processing system 210 obtains an order template indicating a data entity storing information associated with a substance, the data processing system 210 may be configured to identify a substitution group to which the data entity belongs. For example, the data entity may belong to substitution group 230A. The data processing system 210 may be configured to access a data entity substitution configuration of the identified substitution group and update the order template based on the data entity substitution configuration. For example, the data processing system 210 may substitute another data entity in place of the data entity indicated by the order.

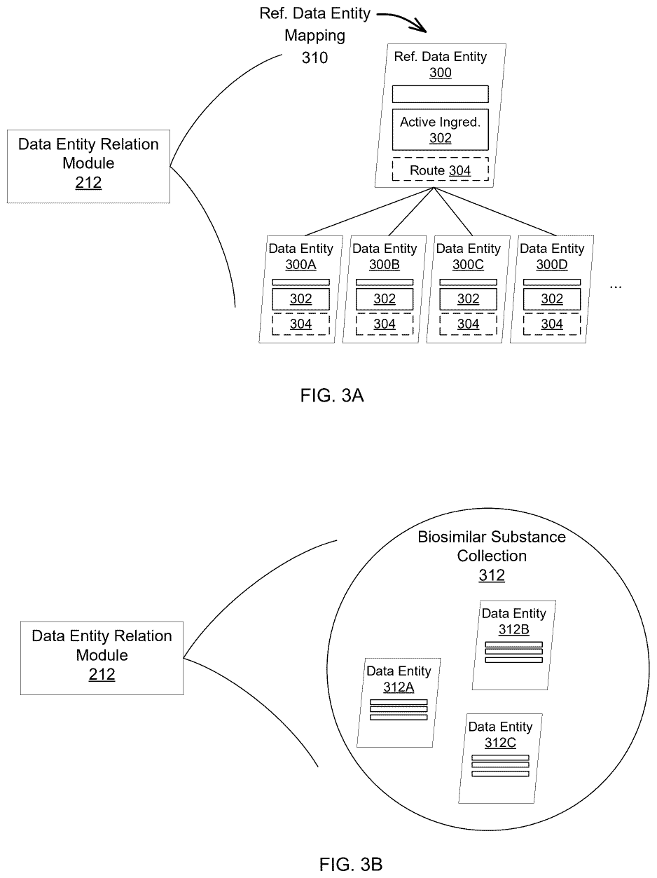

As shown in FIG. 2, the data processing system 210 includes multiple modules including a data entity relation module 212, a substitution group definition module 214, and a substitution module 216. The operation of each module is described herein with reference to FIG. 3A-FIG. 7.

In some embodiments, the order persistence module 204 may be configured to: (1) generate orders 224 using the updated order templates 222B, and (2) persist the orders 224 into the data storage 206. In some embodiments, the order persistence module 204 may be configured to generate an order by reading information from a particular order template and storing it in a data record. For example, the data record may have multiple fields and the order persistence module 204 may store data from the particular order template in appropriate fields of the data record. The order persistence module 204 may be configured to persist the data record in the data storage 206 as an order. In some embodiments, the order persistence module 204 may be configured to store information in an order in addition to information obtained from an applicable order template. For example, the order persistence module 204 may automatically populate fields of a data record from other source(s).

In some embodiments, the order persistence module 204 may be configured to store, in a given order, an indication of modifications made by the data processing system 210 during generation of the order. For example, the order persistence module 204 may include an indication of a substitution of one substance (e.g., one medication) in place of another substance (e.g., another medication) performed by the data processing system 210 (e.g., by modifying an order template based on which the order was generated). When the order is accessed by a user (e.g., through a GUI), the indications of the modifications may be graphically illustrated. For example, a substituted substance may be crossed out in a GUI displaying information about the order. As another example, an overridden delivery method may be crossed out in a GUI displaying information about an order.

In some embodiments, the data entity relation module 212 may be configured to identify sets of related data entities that can be used to define substitution groups. Data entities from a particular set of related data entities may be selected to be included in a substitution group (whereas data entities outside of the set may not be included in the substitution group). For example, a set of related data entities may be a set of data entities associated with substances that share a common active ingredient of a reference data entity. As another example, a set of related data entities may be a set of data entities associated with biosimilar substances. In some embodiments, the data entity relation module 212 may be configured to identify a set of related data entities by: (1) identifying a reference data entity, (2) identifying other data entities that indicate the same active ingredient and active route as the reference data entity, and (3) identifying the reference data entity along with the other data entities as a set of related data entities. For example, the data entity relation module 212 may store a mapping of the reference data entity to the other data entities (e.g., as described herein with reference to FIG. 3A). In some embodiments, the data entity relation module 212 may be configured to identify a set of related data entities by: (1) obtaining an indication of a set of biosimilar substances, and (2) identifying data entities associated with the biosimilar substances as a set of related data entities (e.g., as described herein with reference to FIG. 3B).

FIG. 3A shows an example of the data entity relation module 212 of the data processing system 200 of FIG. 2 mapping a set of data entities to a reference data entity based on a shared active ingredient of substances associated with the set of data entities and the reference data entity, according to some embodiments of the technology described herein. As illustrated in FIG. 3A, the data entity relation module 212 may be configured to access a reference data entity 300 from the data storage 206 of the data record system 200. For example, the data entity relation module 212 may access the reference data entity 300 based on user input indicating a substance associated with the reference data entity 300 (e.g., received through a GUI). The reference data entity 300 indicates an active ingredient 302 of a reference substance associated with the reference data entity 300 (e.g., the reference data entity 300 includes a field storing a value indicating the active ingredient 302). The data entity relation module 212 may be configured to identify other data entities 300A, 300B, 300C, 300D that each indicate the same active ingredient 302 indicated by the reference data entity 300. Accordingly, the reference data entity 300 and the data entities 300A, 300B, 300C, 300D all share a common active ingredient.

In some embodiments, the data entity relation module 212 may be configured to identify data entities that also share a substance (e.g., medication) delivery route 304 with the reference data entity 300 in addition to the active ingredient 302. In the example of FIG. 3A, the data entities 300A, 300B, 300C, 300D indicate the same delivery route 304 as the reference data entity (e.g., each data entity stores a value in a particular field indicating the delivery route 304). In such embodiments, the reference data entity 300 and the data entities 300A, 300B, 300C, 300D indicate both the same active ingredient 302 and the same delivery route 304.

As shown in FIG. 3A, in some embodiments, the data entity relation module 212 may be configured to store a mapping between the reference data entity 300 and the identified data entities 300A, 300B, 300C, 300D. The data entity relation module 212 may be configured to store information (e.g., in data storage 206) mapping the reference data entity 300 to the data entities 300A, 300B, 300C, 300D. For example, the data entity relation module 212 may store, in the reference data entity 300, references (e.g., pointers or links) to the other data entities 300A, 300B, 300C, 300D. As another example, the data entity relation module 212 may store a data record associating the reference data entity 300 with the data entities 300A, 300B, 300C, 300D. The mapping 310 may provide a collection (e.g., a bucket) of data entities represented by the reference data entity 300. The collection of data entities may be used in definition of a substitution group (e.g., as described herein with reference to FIG. 4). Data entities from the collection may be assignable to a substitution group (e.g., to create a substitution group consisting of data entities associated with substances that share a common active ingredient (and optionally a common delivery route)).

In some embodiments, the data entity relation module 212 may be configured to update the mapping 310. If a new data entity is introduced into the data record system 200 and the new data entity indicates the active ingredient 302 (and, optionally, the delivery route 304) indicated by the reference data entity 310, the data entity relation module 212 may add the new data entity to the mapping 310. For example, the data entity relation module 212 may update the information mapping the reference data entity 300 to other data entities to further map the reference data entity 300 to the new data entity. Accordingly, the data entity relation module 212 may keep the mapping 310 up to date with new data entities added to the data record system 210.

FIG. 3B shows an example of the data entity relation module 212 generating a collection 312 of data entities associated with biosimilar substances, according to some embodiments of the technology described herein. In some embodiments, the data entity relation module 212 may be configured to generate the biosimilar substance collection 312 by: (1) obtaining information indicating a set of biosimilar substances (e.g., from the U.S. Food and Drug Administration (FDA)), (2) identifying data entities 312A, 312B, 312C that are associated with the set of biosimilar substances, and (3) generating the biosimilar substance collection 312 that includes the identified data entities 312A, 312B, 312C.

In some embodiments, the data entity relation module 212 may be configured to store a mapping between the data entities 312A, 312B, 312C. The data entity relation module 212 may be configured to store information (e.g., in data storage 206) mapping the data entities 312A, 312B, 312C to each other. For example, the data entity relation module 212 may store a biosimilar substance collection data record specifying the collection of data entities 312A, 312B, 312C. As another example, the data entity relation module 212 may include, in each of the data entities 312A, 312B, 312C, a reference to the other data entities in the biosimilar substance collection 312. The collection of data entities 312 may be used in definition of a substitution group (e.g., as described herein with reference to FIG. 4). Data entities from the collection may be assignable to a substitution group (e.g., to create a substitution group consisting of data entities associated with substances that are biosimilars).

In some embodiments, the data entity relation module 212 may be configured to update the biosimilar substance collection 312. If a new data entity is introduced into the data record system 200 and the new data entity is associated with a substance that is a biosimilar of the substances associated with data entities in the biosimilar substance collection 312, the data entity relation module 212 may add the new data entity to the biosimilar substance collection 312 (e.g., by updating a data record associated with the collection 312). Accordingly, the data entity relation module 212 may keep the mapping biosimilar substance collection 312 up to date with new data entities added to the data record system 210.

FIG. 4 shows an example of the substitution group definition module 214 of the data processing system 200 of FIG. 2 defining a substitution group, according to some embodiments of the technology described herein. As shown in FIG. 4, the substitution group definition module 214 may be configured to provide an indication of related data entities to a user device 402. For example, the substitution group definition module may provide an indication of the reference data entity mapping 310 or an indication of the biosimilar substance collection 312 to the user device 402. In some embodiments, the data entities included in a collection of related data entities (e.g., a biosimilar collection or a collection of a reference data entity with its mapped data entities) may be indicated in a GUI presented in the user device 402. For example, a user of the device 402 may input an identification of the reference data entity 300 (e.g., a name of the substance associated with the reference data entity 300) for a new substitution group and the substitution group definition module 214 may allow the user to add one or more of the data entities 300A, 300B, 300C, 300D mapped to the reference data entity 300 to the substitution group. As another example, a user of the device 402 may input an identification of the biosimilar substance collection 312 (e.g., a name of the biosimilar substance collection) for a new substitution group, and the substitution group definition module 214 may allow the user to select data entities from the collection 312 to the substitution group.

As shown in FIG. 4, the substitution group definition module 214 may be configured to receive user input indicating data entities to assign to the substitution group. In the example of FIG. 4, the substitution group definition module 214 assigns data entities 300A, 300B to the substitution group based on the user input. In some embodiments, the reference data entity 300 may be automatically included in the substitution group 230A (as shown in the example of FIG. 4). In some embodiments, the reference data entity 300 need not be included in the substitution group 230A.

Although in the example of FIG. 4 the substitution group definition module 214 generates the substitution group 230A using a reference data entity mapping, in some embodiments, the substitution group definition module 214 may be configured to generate a substitution group using a biosimilar substance collection of data entities (e.g., biosimilar substance collection 312 described herein with reference to FIG. 3B). For example, the substitution group definition module 214 may receive user input indicating data entities from the biosimilar substance group 312 to assign to a substitution group. The substitution group definition module 214 may assign data entities to the substitution group based on the user input. For example, substitution group 230B may be a substitution group of data entities associated with biosimilar substances.

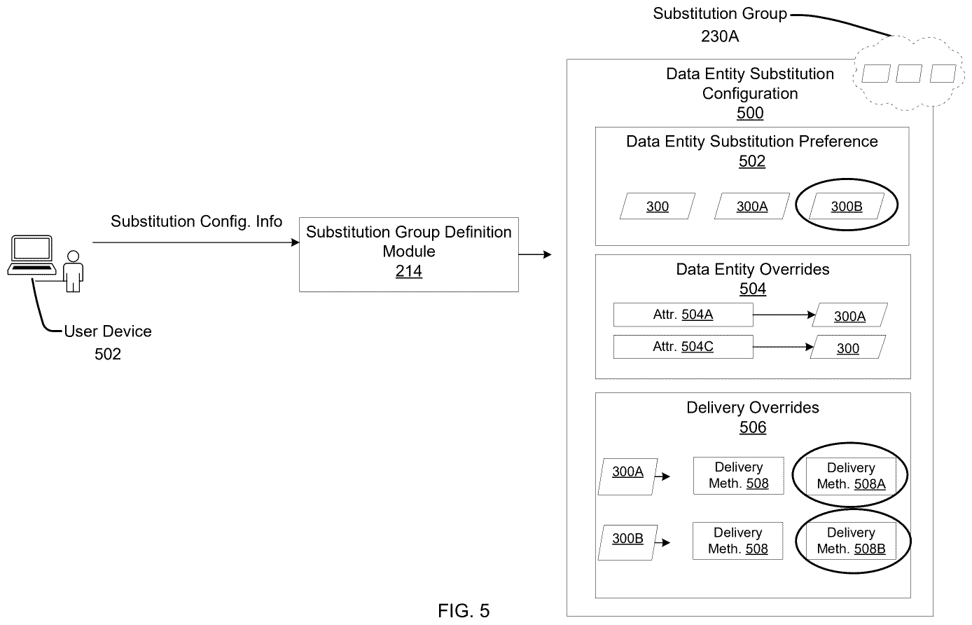

FIG. 5 shows an example of the substitution group definition module 214 generating a data entity substitution group configuration 500 for the substitution group 230A, according to some embodiments of the technology described herein. As shown in FIG. 5, the substitution group definition module 214 may be configured to receive input from a user device 502, where the user input indicates substitution configuration information. For example, the substitution group definition module 214 may provide a GUI through which the user input is provided. The substitution group configuration information may indicate a data entity substitution preference, one or more data entity overrides, and/or a delivery override.

As shown in FIG. 5, the data entity substitution configuration 500 includes a data entity substitution preference 502, data entity overrides 504, and delivery overrides 506.

The data entity substitution preference 502 may indicate a preferred data entity. The preferred data entity may be substituted in place of the other data entities when identified in an order template (assuming there are no overrides). In the example of FIG. 5, the data entity substitution preference 502 indicate that the data entity 300B is the preferred data entity. In some embodiments, the data entity substitution preference 502 may indicate a ranking of data entities associated with different substances. For example, the data entity substitution preference 502 may indicate a ranking of data entity 300B, followed by data entity 300A, and followed by the data entity 300.

In some embodiments, the data entity overrides 504 may indicate instructions to override a preferred data entity indicated by the data entity substitution preferences 502. The data entity overrides 504 may be based on an attribute of a subject (e.g., a patient) associated with an order (e.g., for whom the order is being created). For example, the attribute may be an insurance provider of the subject, a preference indicated by the subject, a diagnosis of the subject, a stage of illness (e.g., a cancer stage) of the subject, or another attribute of the subject. In the example of FIG. 5, the data entity overrides 504 include two override instructions. The first instruction indicates that if a subject has an attribute 504A (e.g., a particular insurance provider), then the data entity 300A is to override the data entity indicated by the data entity substitution preference 502. The second instruction indicates that if a subject has an attribute 504° C. (e.g., another insurance provider), then the reference data entity 300 is to override the data entity indicated by the data entity substitution preference 502.

In some embodiments, the delivery overrides 506 may indicate instructions to override a delivery method associated with a reference data entity storing information associated with a reference substance. A delivery method may specify one or more parameters for administering a substance. For example, the delivery method may specify a fluid amount, a duration of delivery, and/or instructions for the delivery. In the example of FIG. 5, the delivery overrides 506 include an instruction for each of data entities 300A, 300B in the substitution group 230A. For the data entity 300A, the delivery overrides 506 include an instruction to use delivery method 508A instead of the delivery method 508 (which may be the delivery method associated with the reference data entity 300). For the data entity 300B, the delivery overrides 506 include an instruction to use delivery method 508B. In other words, the delivery overrides 506 specify a selection of a delivery method for one or more of the data entities in the substitution group 230A. In some embodiments, the selection for each data entity may indicate a delivery method for a reference substance or a different delivery method for a substance associated with the data entity.

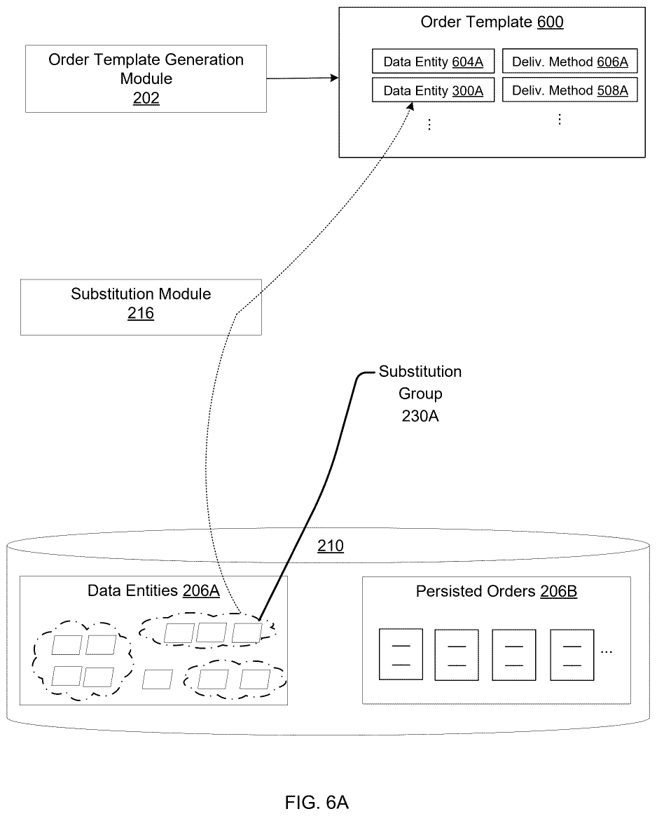

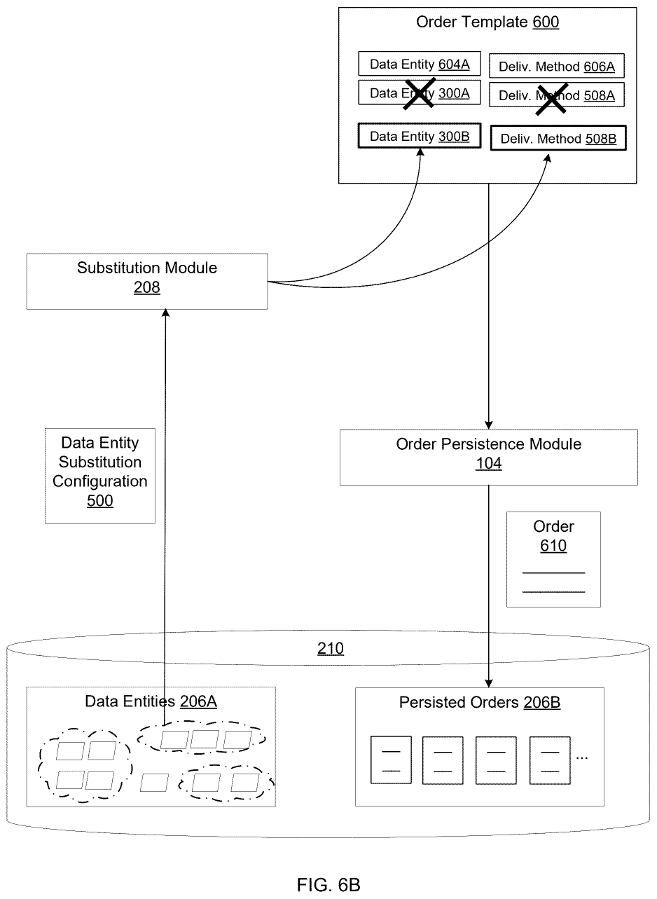

In some embodiments, the substitution module 216 of the data processing system 210 of FIG. 2 may be configured to use the data entity substitution configuration 500 to update an order template indicating a data entity belonging to the substitution group 230A. FIGS. 6A-6B illustrate updating of such an order template 600 performed by the substitution module 216. In FIG.6A-FIG. 6B, an order template 600 is generated by the order template generation module 202 (e.g., in response to user input received from a user device through a GUI provided by the order template generation module to the user device). The order template 600 indicates multiple data entities including data entity 604A and data entity 300A. The order template 600 further indicates a delivery method 606A corresponding to the data entity 604A (for administering the substance associated with data entity 604A) and a delivery method 508A (for administering the substance associated with the data entity 300A).

It should be appreciated that although the order template 600 illustrated in FIG. 6A indicated only data entities and corresponding delivery methods, the order template 600 may indicate other information. For example, the order template 600 may indicate an identity of a subject (e.g., a patient) for whom the order template 600 is generated, an identification of a creator (e.g., a care provider) of the order template 600, administration information, a date, billing information, a diagnosis of the subject, and/or other information.

FIG. 6A illustrates the substitution module 216 determining that data entity 300A indicated by the order template 600 belongs to the substitution group 230A, according to some embodiments of the technology described herein. In some embodiments, the substitution module 216 may be configured to determine that the data entity 300A belongs to the substitution group 230A based on information stored in the data entity 300A. For example, the data entity 300A may include a field storing a value indicating membership to the substitution group 230A. In some embodiments, the substitution module 216 may be configured to determine that the data entity 300A belongs to the substitution group 230A using a record of data entity substitution group memberships. For example, the substitution module 216 may be configured to maintain a record of substitution groups and use the record to determine that the data entity 300A belongs to the substitution group 300A.

FIG. 6B illustrates updating of the order template 600 performed by the substitution module 208, according to some embodiments of the technology described herein. As shown in FIG. 6B, the substitution module 208 may be configured to access the data entity substitution configuration 500 of the substitution group 230A. The data entity substitution configuration 500 indicates a preference to use data entity 300B (as discussed herein with reference to FIG. 5). Accordingly, the substitution module 208 substituted data entity 300B in place of data entity 300A in the order template 600. The data entity substitution configuration 500 further includes a delivery override instruction to set the delivery method for data entity 300B to delivery method 508B. Accordingly, the substitution module 208 substitutes the delivery method 508B in place of the delivery method 508A that was originally in the order template 600.

Although not illustrated in the example of FIG. 6A to FIG. 6B, an order template may indicate a data entity for which an applicable data entity substitution configuration specifies an override instruction based on a subject attribute (e.g., an insurance provider of the subject). In such a case, the substitution module 516 may be configured to update the order template to substitute the overriding data entity in place of the original data entity (instead of a preferred data entity for the substitution group specified by the data entity substitution configuration). For example, the subject for which the order template is generated may have a particular insurance provider. The substitution module 218 may: (1) recognize that the subject has the particular insurance provider, and (2) execute an override instruction associated with an insurance provider attribute indicating the particular insurance provider.

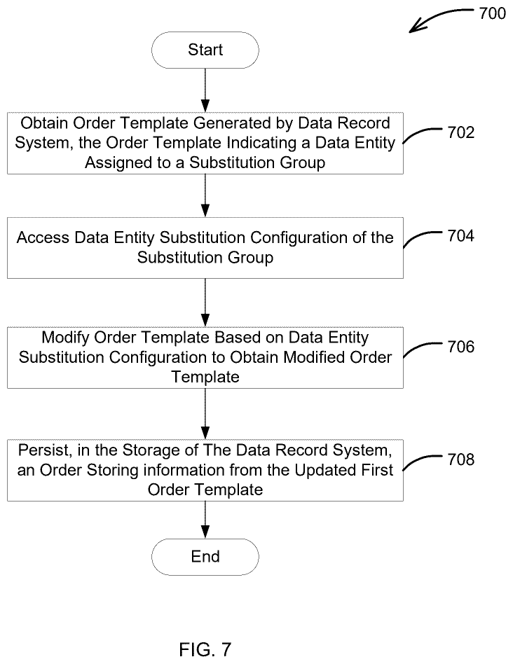

FIG. 7 illustrates a process 700 for automatically substituting information into orders prior to persisting the orders in storage of a data record system, according to some embodiments of the technology described herein. Process 700 may be performed by any suitable computing device. In some embodiments, process 700 may be performed by the data processing system 210 described herein with reference to FIG. 2-FIG. 6B. For example, process 700 may be performed during generation of an order (and prior to persistence of the order in storage). In some embodiments, the system may be configured to perform process 700 automatically such that undesired information in an order is substituted (e.g., one medication is substituted with another preferred medication). Process 700 begins at block 702, where the system performing process 700 obtains an order template (e.g., order template 600) generated by the data record system (e.g., generated by order template generation module 202 described herein with reference to FIG. 2). For example, the order template may be generated by: (1) obtaining information from a user device (e.g., input through a GUI), and (2) populating fields in the order template with the information obtained from the user device. The order template may indicate a data entity that is assigned to a substitution group. For example, the order template may be an order template for a medication order to be persisted in an EHR system. The data entity indicated by the order template may be a data entity storing information associated with a substance (e.g., a medication) to be administered to a subject. In some embodiments, the substance may have an active ingredient (and optionally a delivery route) common to all substances that are associated with data entities assigned to the substitution group. In some embodiments, the substance may be one or multiple biosimilar substances with associated data entities that are assigned to the substitution group.

Next, process 700 proceeds to block 704, where the system accesses a data entity substitution configuration (e.g., data entity substitution configuration 500 of substitution group 230A described herein with reference to FIG. 5) of the substitution group to which the data entity is assigned. In some embodiments, the system may be configured to: (1) identify the substitution group that the data entity is assigned to (e.g., using information in the data entity and/or an external data record of substitution groups and constituent data entities), and (2) access the data entity substitution configuration of the substitution group. For example, the system may be configured to read the data entity substitution configuration from the data storage of the data record system.

Next, process 700 proceeds to block 706, where the system modifies the order template based on the data entity substitution configuration to obtain a modified order template. The data entity substitution configuration may include a data entity substitution preference (e.g., data entity substitution preference 502). The data entity substitution preference may indicate an instruction to substitute a second data entity in place of the data entity. Accordingly, the system may be configured to modify the order template by substituting the original data entity indicated by the order template with the second data entity. In some cases, the data entity substitution configuration may include a data entity override indicating an instruction to override the second data entity specified in the data entity substitution preference with a third data entity. In such cases, the system may modify the order template by substituting the third data entity in place of the original data entity indicated by the order template.

Continuing with the example of a medication order template in an EHR system, the original order template may indicate a first data entity associated with a first medication. The first data entity is assigned to a substitution group of data entities associated with medications that share a common active ingredient or a substitution group of data entities associated with biosimilar medications. In this example, the data entity substitution configuration may include a substitution preference indicating an instruction to substitute the first data entity with a second data entity from the substitution group that is associated with a second medication. Accordingly, the system may substitute the first data entity with the second data entity associated with the second medication. The system may further substitute a delivery method specified for the second data entity in the data entity substitution configuration in place of an original delivery method indicated by the order template.

It should be appreciated that, although in the example of process 700 the order template is modified based on the data entity substitution configuration, it is not necessary that every order template generated by the data record system must be modified. For example, another generated order template may already indicate a preferred data entity specified by a data entity substitution configuration of a substitution group that the other data entity is assigned to. Accordingly, the other order template may not be modified prior to the persistence of an order that incorporates information from the other order template.

In some embodiments, the data entity substitution configuration may include a delivery override indicating delivery methods for various data entities in the substitution group (e.g., one of delivery overrides 506). The system may be configured to modify the order template by modifying which delivery method corresponds to the data entity. The system may be configured to modify the order template such that the delivery method corresponding to the second data entity is the delivery method specified by the delivery override for the second data entity. Accordingly, the modified order template may indicate the second data entity and a corresponding delivery method that was specified by the delivery override.

Next, process 700 proceeds to block 708, where the system persists, in storage of the data record system, an order storing information from the modified order template. In some embodiments, the system may be configured to store information from the modified order template by: (1) accessing information from the second data entity indicated by the modified order template, (2) storing the information in a data record (e.g., in one or more fields of the data record), and (3) persisting the data record in the storage of the data processing system as an order. For example, the system may access medication information stored in the second data entity, store the medication information in one or more fields of a data record, and persist the data record in the storage as an order. In some embodiments, the system may be configured to persist an order that specifies a substance associated with the second data entity (that was substituted into the order template) and a corresponding delivery method. For example, the system may persist an order that specifies a medication associated with the second data entity instead of a medication associated with the data entity originally indicated by the order template. In some embodiments, the system may be configured to further persist an order specifying a delivery method associated with the second data entity. For example, the delivery method may be one that was substituted in place of an original delivery method indicated in the order template obtained at block 702 (e.g., as described herein with reference to FIG. 6B).

In some embodiments, the system may be configured to update a previously persisted order stored in data storage of a data record system. The system may be configured to: (1) recognize that a particular substance (e.g., a medication) indicated in an order is to be substituted with another substance, (2) automatically modifies the order by substituting the substance indicated by the order, and (3) persists the updated order in data storage. The system may be configured to access a substitution group that the particular substance belongs to (e.g., that a data entity associated with the particular substance is assigned to) and modify the order using a data entity substitution configuration of the substitution group. For example, the system may determine, based on the data entity substitution configuration, that the particular substance is to be substituted with another substance (e.g., based on a data entity substitution preference). The system may access information about the other substance from a data entity associated with the other substance and update the order using the information (e.g., by replacing information in the order with the information from the data entity associated with the other substance).

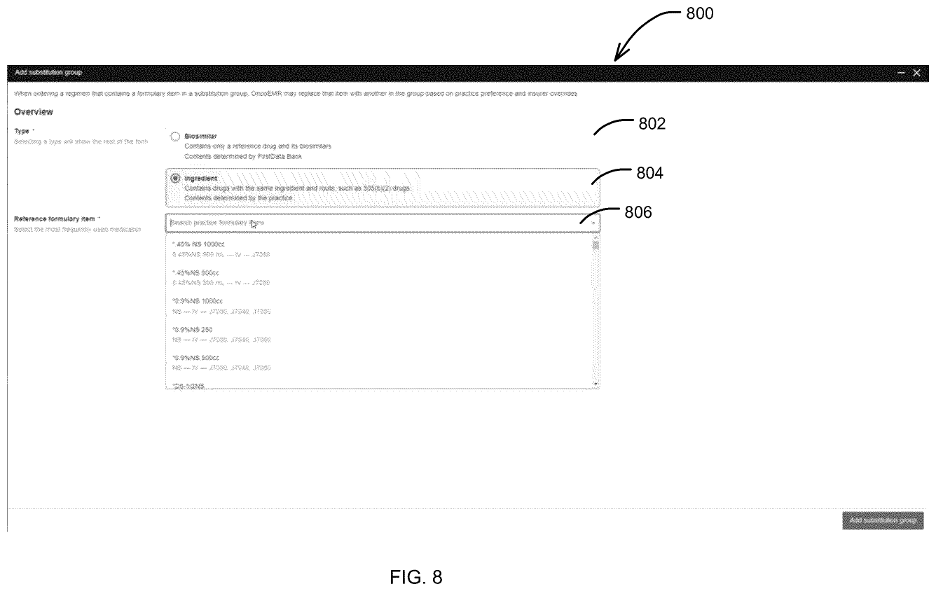

FIG. 8 shows a GUI 800 for generating a new substitution group, according to some embodiments of the technology described herein. The GUI 800 includes an input that allows a user to select a type of substitution group. The options include an option 802 for a substitution group of biosimilar substances and an option 804 for a substitution group of substances sharing a common active ingredient and route. In the example of FIG. 8, the user has selected to generate a substitution group of substances that share a common active ingredient and route. The GUI 800 further includes a field 806 in which the user can specify a reference data entity that can be used to identify other data entities that are assignable to the substitution group (e.g., based on a mapping between the reference data entity and the other data entities).

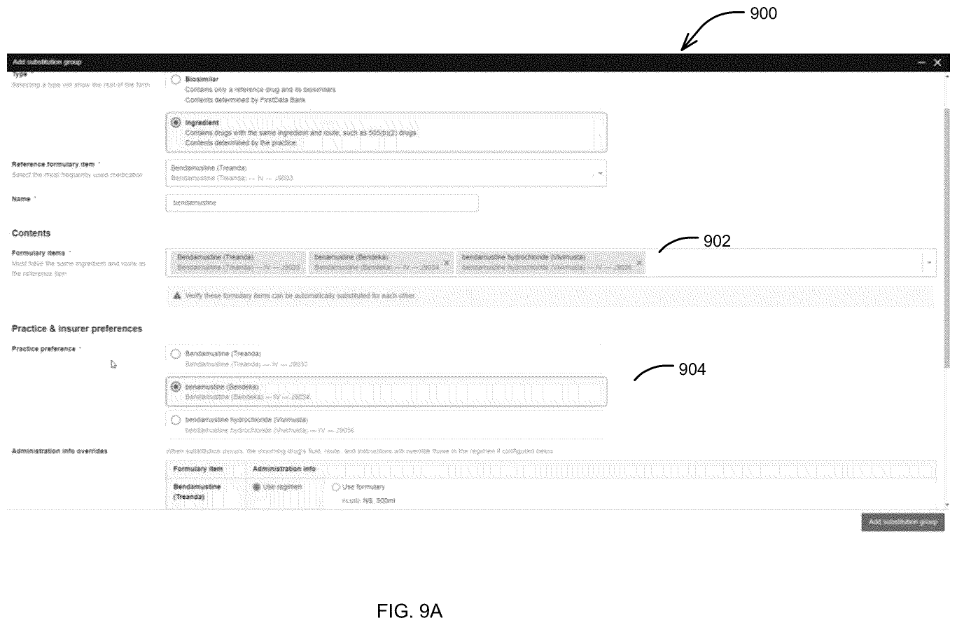

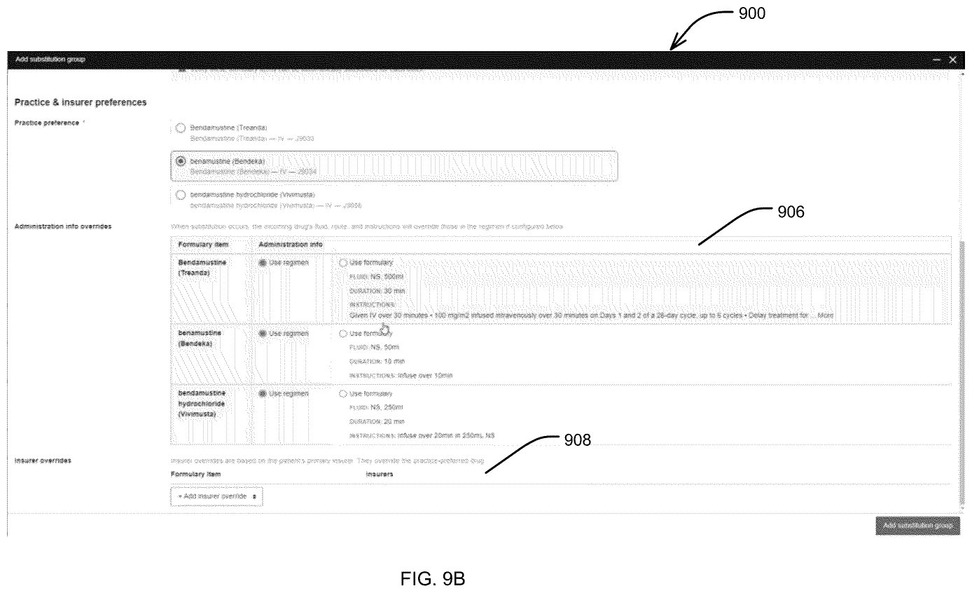

FIG. 9A shows a GUI 900 for specifying a data entity substitution preference of a data entity substitution configuration of a substitution group, according to some embodiments of the technology described herein. The GUI 900 includes a field 902 in which a user may specify data entities associated with substances to include in the substitution group. For example, the system may allow the user to select from among data entities that are mapped to a reference data entity (e.g., as described herein with reference to FIG. 3A-FIG. 3B). The GUI 900 further includes a field 904 that allows the user to specify a preferred data entity from among the data entities added to the substitution group in field 902.

FIG. 9B shows the GUI 900 with a field 906 for specifying delivery overrides of the data entity substitution configuration of FIG. 9A, according to some embodiments of the technology described herein. as shown in FIG. 9B, the field 906 includes an override option for each of the data entities in the substitution group. The field 906 allows the user to select, for each data entity, whether to use a delivery method associated with a reference data entity of the substitution group or an overriding delivery method specific to the data entity. As shown in FIG. 9B, the GUI 900 may include a field 908 to specify one or more data entity overrides based on an insurance provider of a subject.

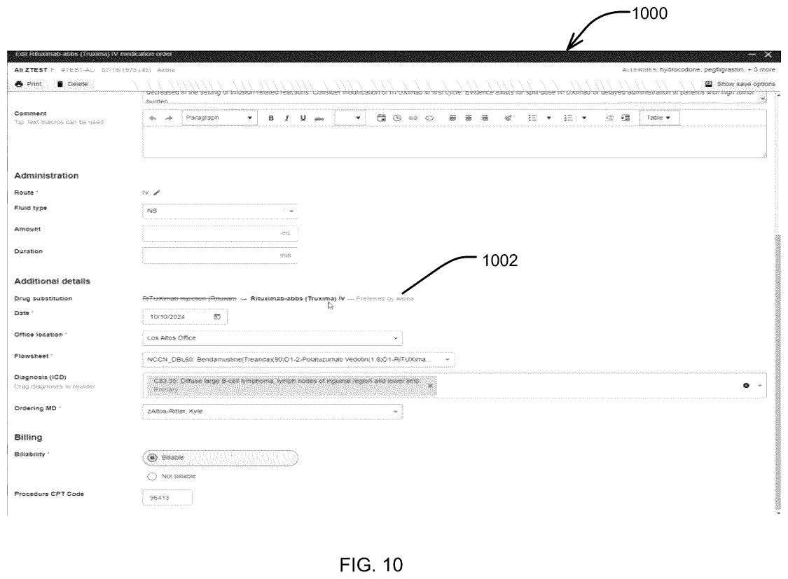

FIG. 10 shows a GUI 1000 for creating an order template, according to some embodiments of the technology described herein. As shown in FIG. 10, the GUI 1000 allows a user to enter various information about an order to be generated based on the order template into various fields. As indicated by the reference label 1002, the system has already substituted a data entity into the order template. Thus, the substituted data entity is the one that the system will draw information from to generate an order from the order template. For example, an order generated from the order template will specify the substance associated with the data entity that was substituted instead of specifying the substance associated with the original data entity that was replaced.

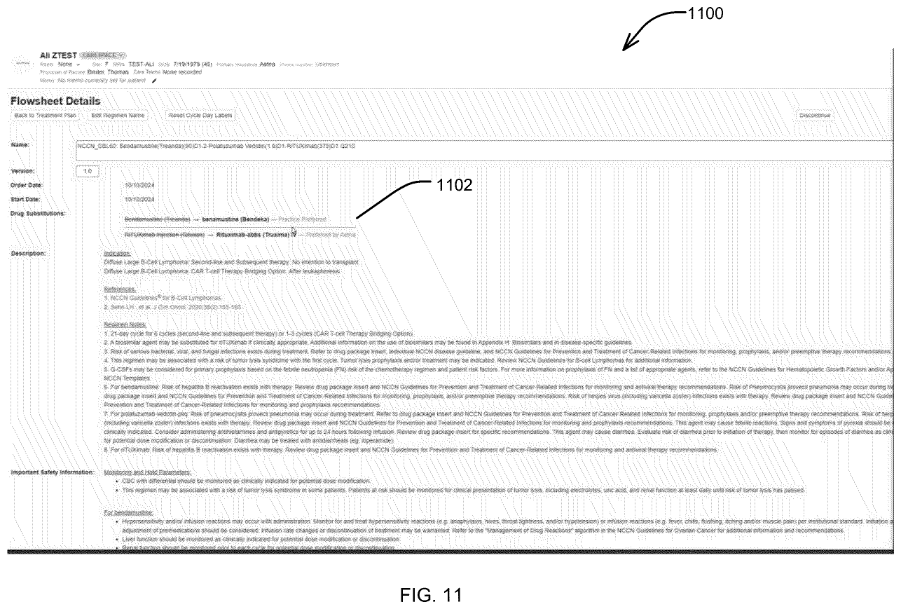

FIG. 11 shows a GUI 1100 displaying information about a persisted order, according to some embodiments of the technology described herein. As indicated by the reference label 1102 FIG. 11, the GUI 1100 indicates that multiple data entities were substituted into the order. The GUI 1100 indicates this by crossing out substances associated with the original data entities and showing an arrow pointing to substances associated with data entities that were substituted into an order template from which the order was generated.

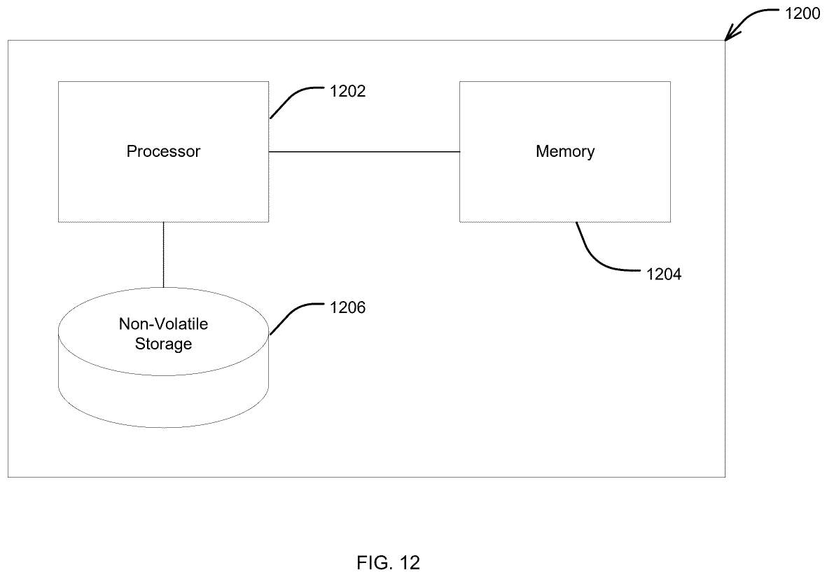

FIG. 12 shows a block diagram of an exemplary computing device 1200 that may be specially configured to implement some embodiments of the technology described herein. For example, the computing device 1200 may be configured to implement the data record system 200 and/or the data processing system 210 described herein with reference to FIG. 2-FIG. 6B.

The computer system 1200 may include one or more computer hardware processors 1202 and non-transitory computer-readable storage media (e.g., memory 1204 and one or more non-volatile storage 1204). The processor(s) 1202 may control writing data to and reading data from (1) the memory 1204; and (2) the non-volatile storage device(s) 1206. To perform any of the functionality described herein, the processor(s) 1202 may execute one or more processor-executable instructions stored in one or more non-transitory computer-readable storage media (e.g., the memory 1204), which may serve as non-transitory computer-readable storage media storing processor-executable instructions for execution by the processor(s) 1202.

The terms “program” or “software” are used herein in a generic sense to refer to any type of computer code or set of processor-executable instructions that can be employed to program a computer or other processor (physical or virtual) to implement various aspects of embodiments as discussed above. Additionally, according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform tasks or implement abstract data types. Typically, the functionality of the program modules may be combined or distributed.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, for example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term). The phraseology and terminology used herein is for the purpose of description and should not be regarded as limiting. The use of “including,” “comprising,” “having,” “containing,” “involving,” and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

What is claimed is:

1. A data processing system for automatically substituting information into orders prior to persisting the orders in storage of a data record system, the data record system storing a plurality of data entities storing data associated with a plurality of substances, the data processing system comprising:
a computer hardware processor; and
memory storing:
data indicating a plurality of substitution groups each assigned a subset of the plurality of data entities and including a data entity substitution configuration, the substitution groups including a first substitution group assigned a first subset of data entities sharing a common active ingredient of respective substances; and
instructions that, when executed by the computer hardware processor, cause the computer hardware processor to:
obtain a first order template generated by the data record system, the first order template indicating a first data entity of the first subset of data entities assigned to the first substitution group;
access a first data entity substitution configuration of the first substitution group, the first data entity substitution configuration indicating an instruction to substitute a second data entity of the first subset of data entities in place of the first data entity;
modify the first order template based on the first data entity substitution configuration at least in part by replacing the first data entity indicated by the first order with the second data entity to obtain a modified first order template; and
persist, in the storage of the data record system, a first order storing information from the second data entity indicated by the modified first order template.

2. The data processing system of claim 1, wherein the computer hardware processor is further configured to generate the first substitution group by performing:
access a reference data entity storing information about a reference substance, the reference data entity indicating a particular active ingredient of the reference substance;
identify, from among the plurality of data entities, data entities based on the reference data entity, the identifying comprising identifying data entities that indicate the particular active ingredient of the reference substance; and
assign at least some of the identified data entities to the first substitution group.

3. The data processing system of claim 2, wherein the reference data entity further indicates a particular delivery route of the reference substance and identifying, from among the plurality of data entities, the data entities based on the reference data entity further comprises:
identifying data entities that indicate the particular delivery route of the reference substance in addition to the particular active ingredient of the reference substance.

4. The data processing system of claim 2, wherein assigning at least some of the identified data entities to the first substitution group comprises:
presenting, in a graphical user interface (GUI), the identified data entities;
receiving, through the GUI, user input selecting the at least some data entities; and
assigning the at least some data entities to the first substitution group in response to receiving the user input through the GUI.

5. The data processing system of claim 2, wherein:
the memory stores data mapping the reference data entity to a set of data entities indicating the particular active ingredient of the reference substance; and
identifying data entities that indicate the particular active ingredient of the reference substance comprises identifying, in the memory, the set of data entities mapped to the reference data entity.

6. The data processing system of claim 5, wherein the computer hardware processor is further configured to:
receive a third data entity storing data associated with a third substance, the third data entity indicating the particular active ingredient of the reference substance; and
after receiving the third data entity, update the data in the memory to map the reference data entity to the third data entity.

7. The data processing system of claim 1, wherein the first data entity substitution configuration further comprises:
a first delivery override, the first delivery override indicating that a first delivery method corresponding to the first data entity is to be overridden by a second delivery method when the second data entity is substituted into an order template.

8. The data processing system of claim 7, wherein the instructions further cause the computer hardware processor to:
modify the first order template based on the first data entity configuration associated with the first substitution group at least in part by changing the first delivery method to the second delivery method.

9. The data processing system of claim 1, wherein the first data entity substitution configuration further includes a data entity override that overrides the instruction to substitute the second data entity in place of the first data entity, wherein the data entity override specifies a particular data entity to use when a target subject in an order template has a particular attribute.

10. The data processing system of claim 9, wherein the instructions further cause the computer hardware processor to:
obtain a second order template generated by the data record system for a first target subject, the second order template indicating the first data entity of the first subset of data entities assigned to the first substitution group;
access the first data entity substitution configuration associated with the first substitution group;
modify the second order template based on the first data entity substitution configuration at least in part by:
determining that the first target subject has the particular attribute specified by the data entity override of the first data entity substitution configuration; and
when it is determined that the first target subject has the particular attribute specified by the data entity override, replacing the first data entity indicated by the second order template with the particular data entity to obtain a modified second order template; and
persist, in the storage of the data record system, a second order storing information from the particular data entity indicated by the modified second order template.

11. The data processing system of claim 1, wherein:
the substitution groups include a second substitution group assigned a second subset of the plurality of data entities that store data associated with a set of biosimilar substances, the second substitution group including a second data entity substitution configuration.

12. The data processing system of claim 11, wherein the instructions further cause the computer hardware processor to perform:
obtain a second order template generated by the data record system, the second order template indicating a third data entity of the second subset of data entities assigned to the second substitution group;
access the second data entity substitution configuration associated with the second substitution group, the second data entity substitution configuration indicating an instruction to substitute a fourth data entity of the second subset of data entities assigned to the second substitution group in place of the third data entity;
modify the second order template based on the second data entity substitution configuration at least in part by replacing the third data entity indicated by the second order template with the fourth data entity to obtain a modified second order template; and
persist, in the data storage of the data record system, a second order storing information from the fourth data entity indicated by the modified second order template.

13. The data processing system of claim 12, wherein the second data entity substitution configuration further includes a second data entity override that overrides the instruction to substitute the fourth data entity in place of the third data entity, wherein the second data entity override specifies a particular data entity to use when a target subject in an order template has a particular attribute.

14. The data processing system of claim 1, wherein the instructions, when executed by the computer hardware processor cause the computer hardware processor to:
update at least one previously persisted order in the storage of the data record system using information from the second data entity indicated by the modified first order template.

15. A method for automatically substituting information into orders prior to persisting the orders in storage of a data record system, the data record system storing a plurality of data entities storing data associated with a plurality of substances, the method comprising:
using a computer hardware processor to perform:
obtaining a first order template generated by the data record system, the first order template indicating a first data entity of a first subset of data entities assigned to a first substitution group of a plurality of substitution groups, the first subset of data entities sharing a common active ingredient of respective substances;
accessing a first data entity substitution configuration of the first substitution group, the first data entity substitution configuration indicating an instruction to substitute a second data entity of the first subset of data entities in place of the first data entity;
modifying the first order template based on the first data entity substitution configuration at least in part by replacing the first data entity indicated by the first order with the second data entity to obtain a modified first order template; and
persisting, in the storage of the data record system, a first order storing information from the second data entity indicated by the modified first order template.

16. The method of claim 15, further comprising generating the first substitution group by performing:
accessing a reference data entity storing information about a reference substance, the reference data entity indicating a particular active ingredient of the reference substance;
identifying, from among the plurality of data entities, data entities based on the reference data entity, the identifying comprising identifying data entities that indicate the particular active ingredient of the reference substance; and
assigning at least some of the identified data entities to the first substitution group.

17. The method of claim 16, wherein the reference data entity further indicates a particular delivery route of the reference substance and identifying, from among the plurality of data entities, the data entities based on the reference data entity further comprises:
identifying data entities that indicate the particular delivery route of the reference substance in addition to the particular active ingredient of the reference substance.

18. The method of claim 16, wherein assigning at least some of the identified data entities to the first substitution group comprises:
presenting, in a graphical user interface (GUI), the identified data entities;
receiving, through the GUI, user input selecting the at least some data entities; and
assigning the at least some data entities to the first substitution group in response to receiving the user input through the GUI.

19. The method of claim 16, wherein:
identifying data entities that indicate the particular active ingredient of the reference substance comprises identifying, in memory of the data processing system, a set of data entities mapped to the reference data entity.

20. A non-transitory computer-readable medium storing instructions that, when executed by a computer hardware processor, cause the computer hardware processor to perform a method for automatically substituting information into orders prior to persisting the orders in storage of a data record system, the data record system storing a plurality of data entities storing data associated with a plurality of substances, the method comprising:
obtaining a first order template generated by the data record system, the first order template indicating a first data entity of a first subset of data entities assigned to a first substitution group of a plurality of substitution groups, the first subset of data entities sharing a common active ingredient of respective substances;
accessing a first data entity substitution configuration of the first substitution group, the first data entity substitution configuration indicating an instruction to substitute a second data entity of the first subset of data entities in place of the first data entity;
modifying the first order template based on the first data entity substitution configuration at least in part by replacing the first data entity indicated by the first order with the second data entity to obtain an updated first order template; and
persisting, in the storage of the data record system, a first order storing information from the second data entity indicated by the modified first order template.
